(19) United States
(12) Patent Application Publication
(10) Pub. No.: US 2021/0042653 A1
(43) Pub. Date: Feb. 11, 2021

(54) METHOD OF DETERMINING A STATE ENERGY

(71) Applicant: RIVER LANE RESEARCH LTD, Cambridge Cambridgeshire (GB)

(72) Inventors: Stephen BRIERLEY, Cambridge Cambridgeshire (GB); Oscar HIGGOTT, London Greater London (GB); Daochen WANG, London Greater London (GB)

(21) Appl. No.: 16/965,907

(22) PCT Filed: Jan. 29, 2019
(86) PCT No.: PCT/GB2019/050238

§ 371 Date: Jul. 29, 2020

(30) Foreign Application Priority Data
Jan. 30, 2018 (GB) 1801517.2

(51) Int. Cl. G06N 10/00; G06F 1/28

(52) U.S. Cl. CPC G06N 10/00; G06F 1/28

(57) ABSTRACT

A method for determining an energy level of a physical system using a quantum computer, wherein the energy level of the physical system is described by the summation of a plurality of summands. The method comprises performing an energy estimation routine which comprises preparing an ansatz trial state, and estimating an expectation value of each summand respectively. The estimating comprises constructing, based on the arrangement of quantum gates, an initial quantum circuit to operate on the ansatz trial state and further comprises performing a summand expectation value determination sub-routine a plurality of times in an iterative process. The energy estimation routine further comprises summing the expectation value estimates of each summand to determine an estimate for the trial state energy. The method further comprises determining the energy level of the physical system by applying an optimisation procedure to the energy estimation routine.

This disclosure relates to quantum computing, and in particular to methods of determining an energy level of a physical system using a quantum computer.

### BACKGROUND

It is extremely useful in many areas of technology to be able to determine the possible energy states of a physical system such as a molecule or atom. Determining how the energy is likely to change as the system is perturbed allows many molecular properties to be derived. For example, by solving the electronic Schrödinger equation for a number of nuclear geometries, it is possible to construct the potential energy surface (PES) of a molecular system. Knowledge of the PES is hugely important, particularly in the field of chemistry, as it allows scientists to determine, among other things, rates of reactions.

Many current methods of obtaining information about the energy states of physical systems rely on classical computers, which use complicated algorithms to simulate the physical system. However, such methods require an unmanageable amount of computing resources and time. It is possible to simulate systems much more efficiently on a quantum computer than is possible on a classical computer, and there has been progress in the experimental development of quantum computers using a variety of architectures. Devices based on trapped-ions and superconducting systems are now above the threshold for fault tolerant quantum computation, meaning that the key building blocks required to scale up to large-scale fault-tolerant quantum computation have now been demonstrated.

To gain an understanding of the disadvantages of prior methods, it is useful to consider the current state of the art of quantum computing, and in particular to consider the coherence time, T, and the maximum circuit depth, D, which today's quantum computers can provide. The maximum quantum circuit depth D relates directly to the coherence time T of the quantum computer. The required circuit depth of an algorithm can be thought of as a factor which quantifies the difficulty of the problem to be calculated. For calculations in which quantum circuit gates can be executed in parallel, the depth of the circuit is the maximum path length between the input and the output of the circuit. The coherence time, in the context of a quantum computer, describes how the environment affects the qubit system. A longer coherence time implies that quantum states can be kept stable for a longer period of time, meaning quantum circuits with increasing depth can be supported, and therefore meaning that more complicated quantum computations can be performed. A quantum computer cannot perform a particular calculation if the circuit depth required by the calculation is too long to be supported by the quantum computer's coherence time.

There are already some known methods which can, in theory at least, be performed on a quantum computer to determine the energy levels of a physical system. Known methods include the Variational Quantum Eigensolver (VQE) method and the Quantum Phase Estimation (QPE) method. However, these known methods have several disadvantages.

VQE can be used to estimate the energy levels of a physical system to a specified accuracy, given knowledge of the Hamiltonian of the system. To perform VQE, a quantum computer need only support a circuit depth of D=O(1). However, to find a state energy of a physical system to a specified accuracy ∈ using VQE, the quantum computer must perform N=O(1/∈²) iterations of quantum circuits.

In other words, in the regime of VQE, the required circuit depth and required coherence time required are relatively small. That means that today's quantum computers can begin to explore physical systems using VQE. However, the number of iterations required for a useful estimate, i.e. one which is reasonably accurate, is prohibitively large. The VQE method therefore has only limited applications, and the results which can be determined take a prohibitively long time to both acquire and process.

In contrast, to find the ground state energy of a Hamiltonian to a specified accuracy ∈ using quantum phase estimation (QPE) on a quantum computer, the quantum computer must perform N=O(log(1/∈) iterations of quantum circuits and is required to support a circuit depth of L)=O(1/∈). QPE therefore requires a reduced number of iterations in comparison with VQE, resulting in potentially faster computations. However, a much longer maximum circuit depth is required. As such, a quantum computer with a very large coherence time is required. In practice, demands on accuracy mean that current quantum computers, and quantum computers which may be built in the foreseeable future, simply cannot provide coherence times which will allow QPE to be performed.

The present invention seeks to address these and other disadvantages of known methods by providing an improved method of determining an energy level of a physical system using a quantum computer.

### SUMMARY

According to an aspect, there is provided a method for determining an energy level of a physical system using a quantum computer. The energy level of the physical system can be described by the summation of a plurality of summands. The method comprises performing an energy estimation routine. The energy estimation routine comprises preparing an ansatz trial state using an arrangement of quantum gates, the ansatz trial state having a trial state energy dependent on a trial state variable. The energy estimation routine also comprises estimating an expectation value of each summand respectively, the estimating comprising constructing, based on the arrangement of quantum gates, an initial quantum circuit to operate on the ansatz trial state and performing a summand expectation value determination sub-routine a plurality of times in an iterative process. The energy estimation routine further comprises summing the expectation value estimates of each summand to determine an estimate for the trial state energy. The method further comprising determining the energy level of the physical system by applying an optimisation procedure to the energy estimation routine, the optimisation procedure comprising iteratively updating the trial state variable and performing the energy estimation routine a plurality of times to determine a respective trial state energy for each of a plurality of different ansatz trial states.

Each iteration of the summand expectation value determination sub-routine may comprise constructing a new quantum circuit, and operating the newly constructed quantum circuit on the ansatz trial state to obtain a measurement value associated with an estimate of the summand expectation value. The new quantum circuit in each iteration of the summand expectation value determination sub-routine may be constructed based on the obtained measurement value. Optionally, the quantum computer has an associated coherence time, T, and the new quantum circuit in each iteration of the summand expectation value determination sub-routine is constructed based on the coherence time.

Constructing new quantum circuits within the summand expectation value determination sub-routine in this manner is in sharp contrast to existing standard VQE summand expectation value determination sub-routines, in which the same quantum circuit operates on a trial state many times. Constructing new quantum circuits in this manner, in particular where circuits are constructed based on the available coherence time, mean that the available coherence time can be maximally exploited as will be discussed in further detail herein.

Each iteration of the summand expectation value estimation sub-routine may further comprise generating a distribution based on the measurement value, and the iterative process may comprise updating the distribution with each iteration based on the mean and standard deviation of the distribution of the previous iteration. This may comprise discarding previous distributions and creating new distributions with each iteration. Estimating the expectation value of each summand may comprise determining the mean of the distribution produced during a final iteration of the summand expectation value determination sub-routine, the sub-routine being performed a predetermined number of times.

Iteratively updating a distribution with each iteration in this manner means that the summand expectation value can be estimated to a given accuracy with a reduced number of iterations. Again, this is in contrast to standard VQE methods for a number of reasons. In standard VQE methods, rather than updating a distribution with each iteration based on the mean and standard deviation of the distribution of the previous iteration, a single distribution is updated with measurement outcomes using a statistical sampling approach.

Optionally, the summand expectation value determination sub-routine comprises operating the quantum circuit on the trial state to obtain a value, μ, associated with the estimate of the expectation value of the summand; determining an error, σ, associated with the value associated with the estimate of the expectation value; and constructing a new quantum circuit based on at least one of the determined error, σ, and the current value of μ. Optionally, the energy level of the physical system is determined to a required accuracy ∈, and the new quantum circuit in each iteration of the summand expectation value sub-routine is constructed based on the required accuracy, ∈. Optionally, the new quantum circuit in each iteration of the summand expectation value sub-routine is constructed with a complexity dependent on T and ∈, T being the coherence time associated with the quantum computer, and the dependence of the complexity of the new quantum circuit on T and ∈ is given by α, wherein:

$$α = ( min ( \frac{log ( T )}{log ( \frac{1}{ϵ} )} , 1 ) .$$

The ability to discard and create new quantum circuits in the summand expectation value determination sub-routine, each newly created circuit having a complexity based on the available coherence time and the required accuracy in the estimate, means that full advantage is taken of available resources.

Discarding quantum circuits associated with previous iterations and producing new quantum circuits as part of an iterative process, in particular when the new quantum circuits are based on parameters determined by operating the previous quantum circuit on the trial state, is completely at odds with the current direction of research in the field of standard VQE methods. In particular, as will be discussed in greater detail herein, creating new quantum circuits by taking into account the available coherence time allows the available resources to be exploited. This is particularly important considering the envisaged future development of quantum computers having longer coherence times.

Optionally, the energy level is determined to a required accuracy, ∈, and the summand expectation value determination sub-routine is repeated N times, wherein N is dependent on ∈.

Optionally, the summand expectation value determination sub-routine is repeated N times, wherein N is based on a coherence time, T, associated with the quantum computer. Again, basing N on the available coherence time allows available resources to be maximally exploited, providing a more efficient method.

Optionally, determining the energy level of the physical system comprises identifying the lowest determined trial state energy. The optimisation procedure may comprise finding a local minimum of the function E(λ).

Optionally, the trial state variable is updated so as to bring the trial state energy of the next ansatz trial state closer to the energy level of the physical system. This is advantageous because, when the trial state energy is equal to the energy state of the physical system of interest, the determination of the trial state energy is equivalent to a determination of the energy state.

Optionally, on a first time the energy estimation routine is performed, the trial state is prepared using the Hamiltonian of the physical system and/or knowledge of the possible states which may be efficiently prepared using the quantum computer.

Optionally, the optimisation procedure comprises repeating the energy estimation routine a plurality of times in an iterative process to determine the energy level of the physical system.

Optionally, the optimisation procedure determines a new trial state variable to be used in the next iteration of the energy estimation routine.

Optionally, each summand comprises an operator, optionally wherein the operator is a tensored Pauli matrix.

According to another aspect, there is provided a computer readable medium comprising computer-executable instructions which, when executed by a processor, cause the processor to perform the method of any preceding claim.

An additional aspect of the invention comprises a method for determining a state energy of a physical system using a quantum computer, the state energy of the physical system being described by the summation of a plurality of summands. The method comprises performing an energy estimation routine comprising preparing a trial state using an arrangement of quantum gates, the structure of the trial state being dependent on a trial state variable. The method may also comprise estimating an expectation value of each summand respectively, the estimating comprising constructing an initial quantum circuit and performing a summand expectation value determination sub-routine a plurality of times in an iterative process. The energy estimation routine may further comprise summing the expectation value estimates of each summand to determine an estimate for the state energy, E, and updating the trial state variable. The energy estimation routine may be repeated a plurality of times in an iterative process to determine the state energy of the physical system.

An additional aspect of the invention comprises a method for determining a state energy, E, of a physical system using a quantum computer. The method comprises performing a trial state energy determination routine comprising preparing a trial state using an arrangement of quantum gates, the trial state being associated with a trial state energy which is dependent on a trial state variable, wherein the trial state energy can be described by the summation of a plurality of summands; determining the expectation value of each summand respectively by performing an iterative summand expectation value determination sub-routine; and summing the determined expectation values to determine the trial state energy, the energy being a function of the trial state variable; and updating the trial state variable. The method may further comprise performing the energy determination routine a plurality of times to obtain a plurality of trial state energy values, and determining the state energy, E, of the physical system by analysing the plurality of determined trial state energy values using an optimisation process.

### FIGURES

Specific embodiments are now described, by way of example only, with reference to the drawings, in which:

FIG. 1 depicts a quantum circuit as known in the prior art;

FIG. 2 depicts the ‘standard’ variational quantum eigensolver approach;

FIG. 3 depicts a quantum circuit for performing rejection filtering phase estimation;

FIG. 4 depicts a circuit used in methods of the present invention for obtaining expectation value estimates;

FIG. 5 depicts a method in accordance with the present invention for determining the state energy of a physical system;

FIG. 6 is a graph which justifies mathematical assumptions made during mathematical derivations presented herein;

FIG. 7 is a plot showing a numerical simulation of a method of the present disclosure, demonstrating the advantages of the method over a method of the prior art;

FIG. 8 is a plot showing a numerical simulation of a method of the present disclosure, demonstrating the advantages of the method over a method of the prior art;

FIG. 9 is a flowchart of a method of determining an expectation value of a summand in accordance with the present invention;

FIG. 10 is a flowchart showing a method according to the present invention.

FIG. 11 is a computer architecture which may be used to perform the methods of the present invention.

### DETAILED DESCRIPTION

This disclosure relates to quantum computing, and in particular to methods of determining an energy level of a physical system using a quantum computer. The energy values of physical systems can generally be described using the Schrödinger equation and via knowledge of the relevant Hamiltonian operator. Accordingly, the disclosure more broadly relates to determining an eigenvalue of a Hermitian operator, in particular the Hamiltonian energy operator, using a quantum computer.

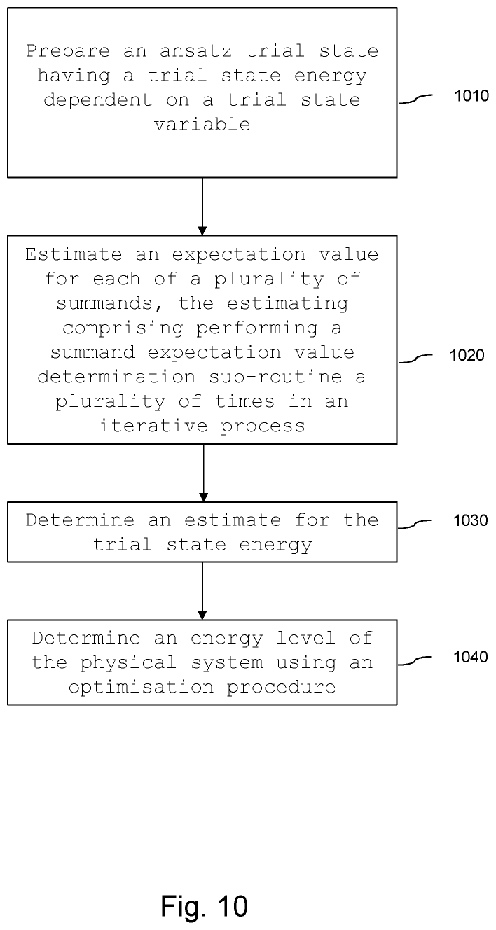

The method of the present disclosure is depicted in the flowchart of FIG. 10. At 1110, an ansatz trial state is prepared. The ansatz trial state has a trial state energy which is dependent on a trial state variable, A. At 1120, an estimate of the expectation value of each of a plurality of summands is obtained. The energy level of the physical system can be described by the summation of a plurality of such summands. Hence, by determining an expectation value of each summand, the energy level, or state, of the physical system can be determined. The estimating comprises performing a summand expectation value determination sub-routine a plurality of times in an iterative process. The introduction of an iterative sub-routine to a summand expectation value sub-routine within the frame work of VQE in this manner has never before been considered by practitioners of VQE. The iterative sub-routine will be detailed in greater detail herein.

At 1130, an estimate for the trial state energy is determined. This determination is based on the expectation values obtained from step 1120. Finally, at 1140, an energy level, or state, of the physical system is determined using, or according to, an optimisation procedure. The optimisation procedure may comprise preparing and discarding quantum states, and the method may comprise performing steps 1110, 1120, and 1130 a plurality of times as will be described in greater detail herein.

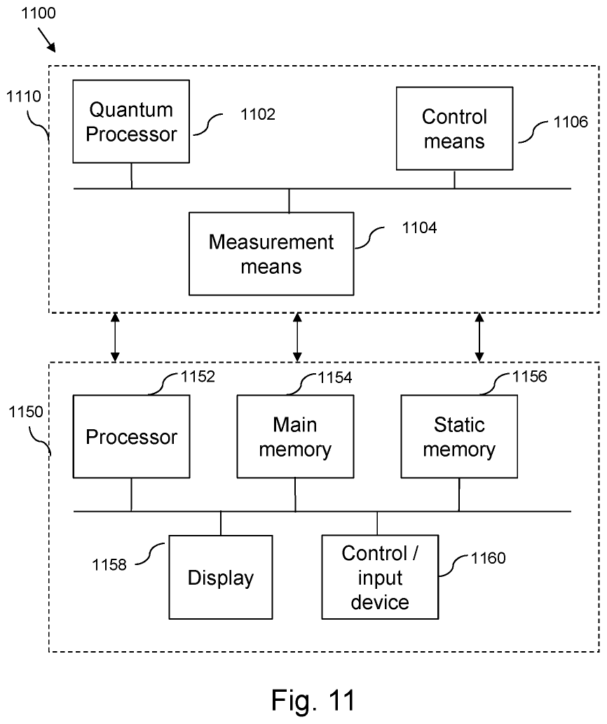

FIG. 11 illustrates a block diagram of one implementation of a computing device 1100 within which a set of instructions for causing the computing device to perform any one or more of the methodologies of the present disclosure may be executed. While only a single computing device is illustrated, the term “computing device” shall also be taken to include any collection of machines (e.g., computers) that individually or jointly execute a set (or multiple sets) of instructions to perform any one or more of the methodologies discussed herein. The computing device 1100 comprises a quantum computing system 1110 and a classical computing system 1150. The quantum computing system 1110 is in communication with classical computing system 1150. The classical computing system is arranged to instruct the quantum computing system to prepare quantum states, and to perform measurements on those quantum states, according to instructions stored in memory.

The quantum computing system 102 comprises a quantum processor 1102, which in turn comprises at least two qubits and at least one coupler capable of coupling the qubits. The qubits may be physically implemented using, for example, photons, trapped ions, electrons, one or more nuclei, superconductor circuits and/or quantum dots. In other words, a qubit may be be physically implemented in a variety of means, including the polarization state of a single photon; the spatial optical path of a single photon; two differing energy states of an atom or an ion; the spin orientation of a particle or plurality of particles such as a nucleus. The quantum computer also comprises means for storing the qubits and maintaining the qubits in a suitable environment to allow quantum computation, for example means for supercooling the qubits. The qubits may be operated upon by one or more quantum circuits, formed by a suitable arrangement of quantum gates.

A quantum gate acts on some number of qubits and can be thought of as the quantum analogue of a basic low-level instruction in a classical circuit such as a NOT or AND gate. Typically, quantum circuits are decomposed into a sequence of single and two-bit gates taken from a universal gate set along with state preparation and the measurement or read-out of the qubits. The results of the measurements are classical data that are then processed by a classical computer. Many quantum computers based on superconducting circuits and trapped-ions have already demonstrated all of the capabilities at a small scale that are required for a large quantum computing device.

Possible implementations and methods of manipulation of the qubits in the quantum computer are now described. These implementations are by way of example only, and the skilled person will be aware of other methods of implementing a quantum computer. Birefringent wave plates may be used to manipulate the polarization state of a single photon, for example, to cause a linear polarization or horizontal polarization of the photon, signifying two distinct states of the photon. The qubits may also be implemented using a beam splitter. For example, the presence or absence of a photon along particular optical path can be implemented using a beam splitter that splits a beam of photons into two separate paths. The presence of the photon in either path represents two distinct states of the photon. Alternatively or additionally, two separate electronic energy states for an atom or ion can represent two separate distinct states for a qubit. For example, transition energies between these levels may correspond to the energy of electromagnetic radiation of a certain frequency and so the separate energy states of the atom or ion may be addressed using a source of radiation such as a laser or microwave emitter. Alternatively or additionally, the two distinct spin states (spin “up” and spin “down”) of a particle or a plurality of particles, for example a nucleus, can represent the two distinct states of a qubit. Manipulations of nuclear spin may be implemented using a magnetic field using methods known to the person skilled in the art.

Alternatively or additionally, superconducting electronic circuits may be used to create qubits. These systems are supercooled to below 100K and use Josephson junctions, a non-linear inductor that allows the creation of anharmonic oscillators. Anharmonic oscillators do not have evenly spaced energy levels (unlike harmonic oscillators) and therefore two of the states can be separately controlled, and used to store a qubit. The qubits are connected with microwave cavities and single and two-qubit gates can be performed using microwave signals.

The quantum computing device 1110 also comprises measurement means 1104 and control means 1106. The control means 1106 may comprise control hardware and/or a control device. The control means 1106 is configured to receive instructions from the classical computer 1150, and the classical computer 1150 may instruct the control means 1106 to prepare a particular state in the quantum processor using a particular arrangement of quantum gates. The measurement means 1104 may comprise measurement hardware and/or a measurement device. The measurement means comprises hardware configured to take a measurement from a state prepared by the control means 1106 in the quantum processor 1102.

The example classical computing device 1150 includes a processor 1152, a main memory 1154 (e.g., read-only memory (ROM), flash memory, dynamic random access memory (DRAM) such as synchronous DRAM (SDRAM) or Rambus DRAM (RDRAM), etc.), a static memory 1156 (e.g., flash memory, static random access memory (SRAM), etc.), and a secondary memory (e.g., a data storage device), which communicate with each other via a bus.

Processing device 1152 represents one or more general-purpose processors such as a microprocessor, central processing unit, or the like. More particularly, the processing device 1152 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1152 may also be one or more special-purpose processing devices such as an application specific integrated circuit (ASIC), a field programmable gate array (FPGA), a digital signal processor (DSP), network processor, or the like. Processing device 1152 is configured to execute the processing logic for performing the operations and steps discussed herein.

The data storage device may include one or more machine-readable storage media (or more specifically one or more non-transitory computer-readable storage media) on which is stored one or more sets of instructions embodying any one or more of the methodologies or functions described herein. The instructions may also reside, completely or at least partially, within the main memory 1154 and/or within the processing device 1152 during execution thereof by the computer system, the main memory 1154 and the processing device 1152 also constituting computer-readable storage media.

In general, the classical computer 1150 instructs the control means 1106 of the quantum computer 1110 to prepare a particular state in the quantum processor 1102. The control means 1106 manipulates the qubits in the quantum processor 1102 based on the instructions. Once the qubits have been manipulated such that the desired state has been constructed in the quantum processor 1102, the measurement means 1104 takes a measurement from the state. The quantum computer 1110 then communicates the measurement result to the classical computer.

The various methods described herein may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying,” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The prior art methods, ‘standard QPE’ and ‘standard VQE’ are now briefly discussed.

Standard QPE

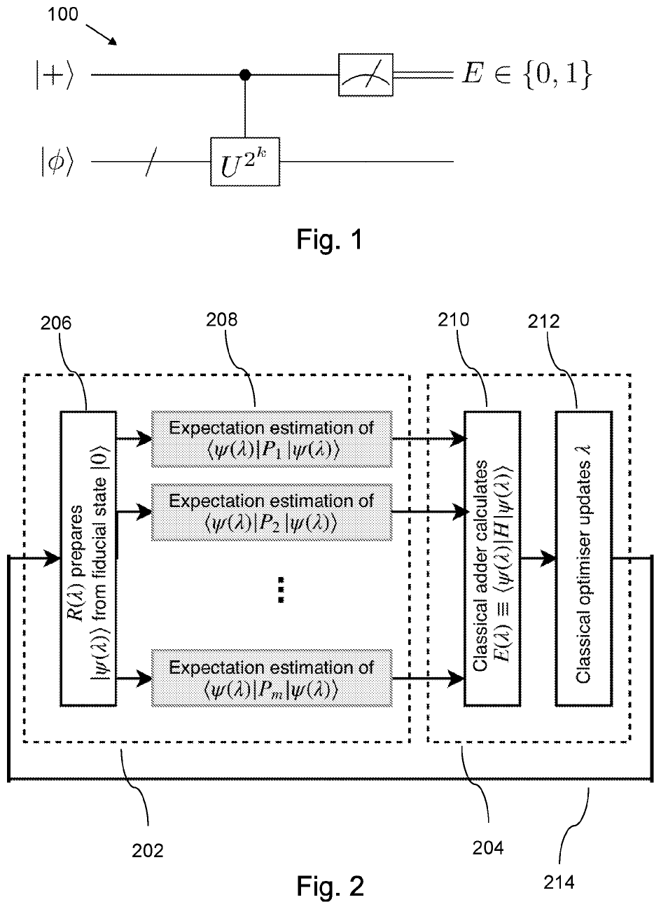

FIG. 1 shows a schematic circuit 100 which may be used as part of standard QPE methods. Since the introduction by Kitaev of a type of iterative QPE involving a single work qubit and an increasing number of controlled unitaries at each iteration, the term “QPE” has become associated with algorithms of this particular type. It is characteristic of Kitaev-type algorithms that for precision ∈, the number of iterations N=O(log(1/∈)) and maximum quantum circuit depth D=O(1/∈), where the tilde means we neglect polylog factors. This neglect is justified not only because polylog factors are small but because there exist Kitaev-type algorithms where they are (essentially) eliminated, e.g. Information Theory Phase Estimation (ITPE) replaces an extra log log(1/∈) in Kitaev's QPE by log*(1/∈)

Henceforth, Kitaev-type scaling

$$N = O ( log ( \frac{1}{ϵ} ) ) , D = O ( 1 / ϵ )$$

is referred to as the phase estimation regime and QPE as phase estimation in this regime.

QPE has found application in quantum chemistry where it can be used to estimate the ground state energy of a chemical Hamiltonian. However, the required circuit depth depends on accuracy as follows: D=O(1/∈), which implies that a very large coherence time is required to obtain accurate results.

Standard VQE

Reference is now made to FIG. 2, which depicts a known method of determining the energy level of a physical system. The known method is referred to as the variational quantum eigensolver (VQE) approach. Dashed box 202 depicts those parts of the method which are performed using a quantum computer, using quantum circuits. Dashed box 204 depicts those parts of the method which are performed using a classical computer, using classical circuits. Arrows between dashed boxes 202 and 204 depict the interface between the quantum and classical computers.

As will be understood by the skilled person, the energy states of a physical system may be described using a Hamiltonian operator. The standard VQE method can be used to determine the ground state energy of a Hamiltonian H of a physical system using a quantum expectation estimation sub-routine together with a classical optimizer. The classical optimizer adjusts the energy of variational ansatz wavefunctions |ψ(λ) [symbol] , depending on a parameter λ. For a given normalized |ψ(λ)>, it is possible to evaluate energy:

E(λ)≡ [symbol] ψ(λ)|H|ψ(λ) [symbol] =Σaᵢ [symbol] ψ(λ)|Pᵢ|ψ(λ)

[symbol]

To describe the standard VQE in more detail, the idea is to first write the Hamiltonian operator, H, as a finite sum H=ΣaᵢPₜ where aᵢ are complex coefficients and Pᵢ are tensored Pauli matrices. The set of Pauli matrices forms a basis for the space in which H belongs. Each aᵢPᵢ can be described as a summand. The number m of summands is assumed to be polynomial in the size of the system as is the case for the electronic Hamiltonian of quantum chemistry.

To evaluate the energy state of the physical system, knowledge of the Hamiltonian is used to determine an ansatz trial state. This ansatz trial state has an energy E(λ), dependent on a parameter λ. The trial state is prepared in the quantum processor, and quantum circuits 202 are used to determine the expectation values of each summand. Given the expectation value estimates, a classical computer 204 is used to determine the weighted sum. This summation produces an estimate and/or a determination of the trial state energy. Finally, a classical gradient-free optimiser such as Nelder-Mead is used to optimise the function E(λ) with respect to λ by controlling a preparation circuit:

R(λ):|0 [symbol] →|ψ(λ)

[symbol]

where |0 [symbol] is a fiducial starting state. The variational principle (VP) justifies the entire VQE procedure when finding the ground state: writing Eₘᵢₙ for the ground state eigenvalue of H, VP states that E(λ)≥Eₘᵢₙ with equality if and only if |ψ(λ)> is the ground state. Similarly, local minima are representative of other energy levels/states of the physical system.

In the typical VQE process, a preparation circuit, R, comprised within the quantum computer is used to prepare an initial trial state |ψ(λ) [symbol] . The preparation of the initial trial state is shown at box 206 of FIG. 2.

The expectation value of each term in the Hamiltonian can then be estimated for the given trial state. This determination is shown at blocks 208 of FIG. 2. In other words, to determine an energy eigenvalue of a Hamiltonian with m summands, the quantum computing device measures: [symbol] ψ(λ)|P₁|ψ(λ) [symbol] ; [symbol] ψ(λ)|P₂|ψ(λ) [symbol] ; . . . [symbol] ψ(λ)|Pₘ|(λ) [symbol] for the trial state.

These expectation values are communicated to a classical computing device, depicted by dashed box 204 in FIG. 2. The classical computing device sums the summands together to find the energy eigenvalue of the Hamiltonian for the initial trial state. Based on this eigenvalue, the classical computer 204 updates the parameter λ at box 212, which allows the constructions of a new trial state. The quantum computer is instructed to prepare the new trial state, and the whole process is repeated until an optimisation procedure is satisfied that the desired energy level has been determined to the specified accuracy.

As will be understood by the skilled person, each expectation [symbol] ψ(λ)|Pᵢ|ψ(λ) [symbol] may be directly measured using a simple circuit, or could be measured by using an extra work qubit and a C−Pᵢ gate, which can be implemented by a small circuit involving single qubit gates and C−NOT gates. In both cases, the circuit involved is of D=O(1) depth and is repeated N=O(1/∈²) times to attain precision within ∈ of the expectation. Herein, the regime wherein N=O(1/∈²), D=O(1) is referred to as the statistical sampling regime.

Note that the quantum-over-classical advantage is hidden within the set of ansatz states {|ψ(λ) [symbol] }_{λ}, chosen so that they could always be efficiently prepared on a quantum computer but not usually on a classical computer. The set of Unitary Coupled Cluster (UCC) states is a typical choice and could not usually be efficiently prepared classically due to the non-truncation of the BCH expansion of an operator of form 2^{T-τ}^{^{†}}. Another two possible choices are the device ansatz and adiabatic ansatz.

Importantly, in standard VQE as depicted in FIG. 2, the summands in each of the boxes at 208 are determined using statistical sampling. In other words, the same, simple quantum circuit of depth D=O(1) is operated on the trial state a plurality of times, each time giving a different measurement outcome which is used to populate a single distribution. Operating the same quantum circuit on the trial state many times gives statistical accuracy in the measurement of the summand, however the number of required repetitions is often unfeasibly large, since the required number of repetitions N=O(1/∈²), scales exponentially with required accuracy ∈.

As will be explained in greater detail below, methods of the present disclosure make use of the framework of VQE but are able to determine energy levels in a considerably shorter time than the VQE method by optimizing the method according to the required accuracy and the limitations of the available quantum computer. Importantly, the present method performs a summand expectation value determination sub-routine a plurality of times in an iterative process. The iterative nature of the sub-routine is in stark contrast to the standard VQE method. In each iteration of the presently disclosed sub-routine, a new quantum circuit is created, and the previous circuit is discarded. The new quantum circuits may be created based on the obtained measurement value of the previous circuit. The new circuits may also be created based on the available coherence time, and with each iteration a new distribution may be generated. This is more than simple statistical sampling, as is used in standard VQE methods, and the present method allows the summand expectation value to be determined using quantum circuits of varying lengths and complexities in a manner which maximizes the use of available coherence time.

Methods of the present disclosure will now be described in further detail.

Tunable Bayesian QPE (α-QPE)

In methods of the present disclosure, a new and innovative approach is used to determine the values of each summand. Instead of performing a large number of iterations of the same quantum circuit to achieve a high accuracy, as is required in VQE methods, the summand is calculated using an iterative process. Generally speaking, the iterative process involves constructing a plurality of different quantum circuits. In the iterative process, an initial quantum circuit is constructed. The initial quantum circuit is constructed based on a quantity α which will be defined below.

The initial quantum circuit is constructed based on the coherence time, T, of the quantum computer and/or the quantum processor which is being used to perform the determination. The initial quantum circuit is also constructed based on the required accuracy, ∈, in the determination. The initial quantum circuit operates on a trial state, which is prepared using knowledge of the Hamiltonian of the physical system in question. Each time a quantum circuit operates on the trial state, a measurement outcome is obtained. In particular, in each iteration, a quantum circuit operates on the trial state to obtain a value, μ, associated with the estimate of the expectation value of the summand. An error, σ, in the measurement outcome is also determined, the error being associated with μ. Finally, each iteration of the iterative process involves constructing a new quantum circuit based on the determined error, σ, and the current value of μ.

Importantly, this constructing and discarding of quantum circuits in an iterative manner is completely new to the framework of VQE, and allows an accurate determination of the expectation value with fewer iterations by tailoring the summand expectation value determination sub-routine to the available coherence time. The underlying mathematics of the new method will now be detailed.

For a given eigenvector |ϕ [symbol] of a given unitary operator U such that U|ϕ [symbol] =e^{iϕ}|ϕ [symbol] , φϵ[−π, π) and a required precision ∈, current QPE methods use N=O(log(1/∈)) iterations of circuits involving c−U²^{^{N−1}}, c−U²^{^{N−2}}, . . . , c−U¹, c−U⁰ gates in that order to estimate the phase ϕ to precision ∈ within a constant probability of error. The maximum depth is D=2^{N-1}=O(1/∈) when viewing c−U²^{^{N−1}} as a sequence of 2^{N-1} c−U gates. This is the correct view to relate D to coherence time requirements as in quantum simulation when U is of the form exp(−itH), and separately assuming |ϕ [symbol] is re-prepared at each iteration.

It is to be understood that “precision ∈” means (as appropriate) either the frequentist or Bayesian Δ(\hat{ϕ})≤∈ where Δ(\hat{ϕ}) is either the point estimator or posterior standard deviation respectively. In other words, the meaning of “precision ∈” approximates “accuracy ∈” (i.e. |\hat{ϕ}−ϕ|≤∈) under the assumption of asymptotic consistency.

In contrast to QPE, the expectation estimation algorithm of VQE would estimate ϕ to precision ∈ by statistical sampling using N=O(1/∈²) circuits that are exactly the same which gives D=O(1). In other words, the expectation estimation algorithm of VQE would estimate ϕ to precision E by performing N=O(1/∈²) of the same circuit, that circuit having depth D=O(1). The estimate \hat{ϕ} is a maximum likelihood estimator since it is a function ƒ(\hat{p}) of the relative frequency estimator \hat{p} of a probability p with ϕ=ƒ(p).

In contrast, methods of the present disclosure clow an optimal tradeoff between N and D. This is important in experiments where N is the number of state preparations or the number of measurements and D is proportional to coherence time requirements. The best tradeoff therefore depends on the capabilities of the experimenter's device. The present method relates to a continuous family of circuit sequences giving tradeoffs that interpolate between phase estimation and statistical sampling. Methods of the present disclosure make use of Rejection Filtering Phase Estimation (RFPE).

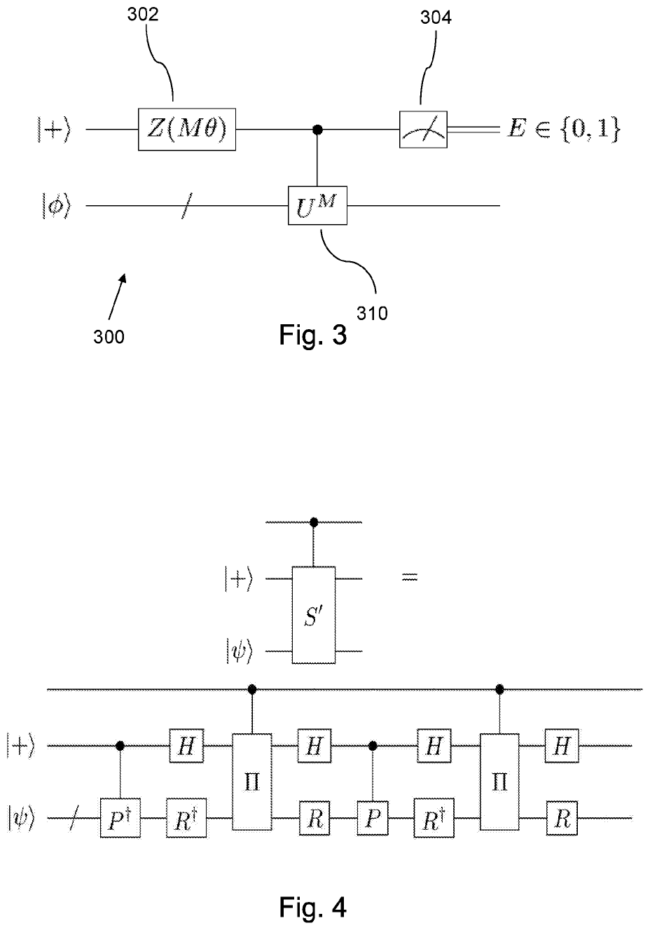

A quantum circuit suitable for use in RFPE is shown schematically in FIG. 3. The quantum circuit 300 comprises a top wire that comprises a rotation operator 302 and a measurement 304. The quantum circuit further comprises a bottom wire wherein the trial state |ϕ [symbol] is operated on by the operator U^{M} 310 conditional on the top wire. The operator U^{M} 310 comprises M applications of U operating on the trial state |ϕ [symbol] .

The rotation operator 302 on the top wire applies a rotation by an angle Mθ in the computational basis to the |+ [symbol] state along the top wire. The |+ [symbol] state is the +1 eigenstate of the tensored X Pauli operator. This qubit is then used to control the operator U^{M} before a measurement 304 is performed on the top wire to obtain a measurement outcome E, where E can be a 0 or a 1.

The result of the measurement outcome is then analysed in order to choose the subsequent values of M and θ with the ultimate goal of determining the unknown quantity ϕ.

To begin, an initial prior probability distribution P(ϕ) of ϕ is taken to be normal [symbol] ((μ,σ²), reflecting any prior knowledge of the solution and then approximated by a normal distribution. Before each iteration of the circuit, M and θ are chosen to minimise the expected posterior variance (i.e. the Bayes risk). A method for achieving this is given in the Appendix. Given the RFPE circuit in FIG. 3 and a prior distribution P(ϕ) of ϕ, the probability of measuring E∈{0,1} is

$$P ( E \vert φ ; M , θ ) = \frac{1 + {( - 1 )}^{E} cos ( M ( φ - θ )}{2} ,$$

which, by the Bayesian update rule, informs the posterior after measuring E:

P(ϕ|E;M,θ)∝P(E|ϕ;M,θ)P(ϕ).

It is not necessary to know the constant of proportionality to sample from this posterior after measuring E, and the word “rejection” in RFPE refers to the rejection sampling method used. After obtaining a number m of samples, the posterior can be approximated by a normal with mean and variance equal to that of the samples. This is justified in the same way as when taking initial prior to be normal. The choice of m is important and m can be regarded as a particle filter number, hence the word “filter” in RFPE. The posteriors are approximated to be normal essentially because this allows for efficient sampling in the next iteration.

The effectiveness of RFPE's iterative update procedure depends on controllable parameters (M,θ). A natural measure of effectiveness is the expected posterior variance, i.e. the “Bayes risk”. To minimise the Bayes risk, standard QPE methods have used

$$M = \frac{1.25}{σ}$$

at the start of each iteration. However, the main problem is that M can quickly become large, making the depth of U^{M} exceed Dₘₐₓ. This issue has previously been partially mitigated by imposing an upper bound on M. This method is hereinafter referred to as RFPE with restarts.

The present disclosure uses a different approach wherein M and θ are chosen as:

$$( M , θ ) = ( \frac{{1}^{α}}{σ} , μ - σ )$$

where α∈[0,1] is a free parameter imposed. Moreover, at each iteration, the eigenstate |ϕ [symbol] may be re-prepared, allowing the state used in the previous iteration to be discarded. This requires the ability to readily prepare an eigenstate on the quantum computer. As discussed above, the trial states are chosen such that they may be efficiently prepared on a quantum computer. The resulting algorithm is hereinafter referred to as the α-QPE algorithm.

As derived in the Appendix, α-QPE requires:

N=ƒ(∈,α),D=1/∈^{α}

wherein the number of iterations/measurements and the maximum coherent depth are given by N and D respectively, and the function ƒ is given by

$$f ( ϵ , α ) = \{ \begin{matrix}\frac{2}{1 - α} ( \frac{1}{{ϵ}^{2 ( 1 - α )}} - 1 ) 0 \leq α < 1 \\ 4 log ( \frac{1}{ϵ} ) α = 1\end{matrix}$$

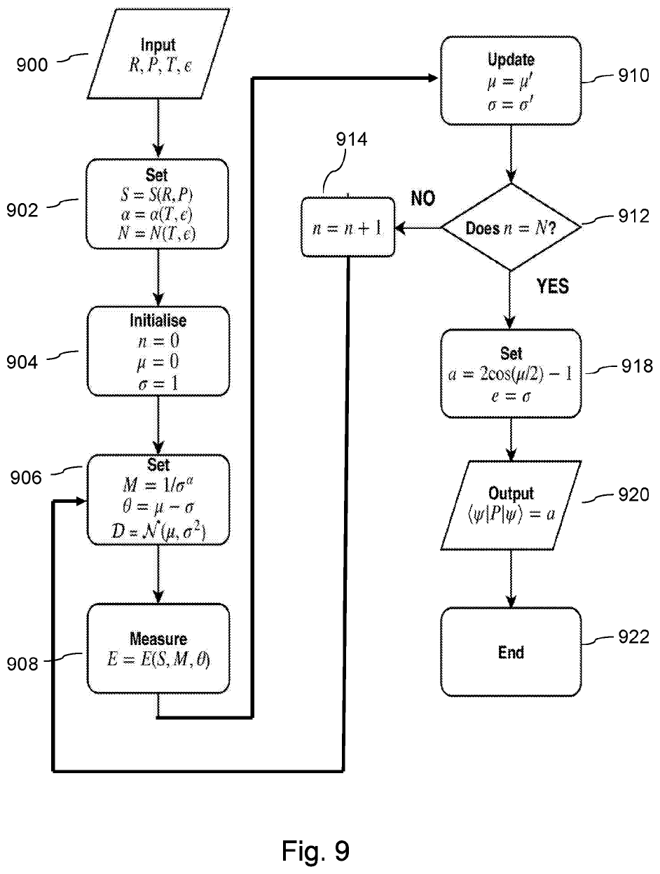

The α-tunable Bayesian QPE of the present method is hereinafter referred to as α-QPE. A flowchart of α-QPE is given in FIG. 9. When making reference to RFPE above, the reference is only to its Bayesian method rather than its specific form of implementation. It is understood that other sequences {U^{M}^{ᵢ}}ᵢ can also be analysed with relative ease using this Bayesian method. More generally, both RFPE and α-QPE are examples of (online, decision theoretic, noisy, Bayesian) active learning algorithms with a quantum device performing labelling. Active learning is expected to be to be highly relevant to hybrid quantum-classical algorithms since it accounts for labelling costs.

Casting Expectation Estimation as α-QPE

As detailed later with respect to the flowchart of FIG. 9, the α-QPE method of the present disclosure may determine the expectation value of a measurement operator, P, corresponding to one of the summands in a Hamiltonian of a physical system, by making use of a preparation circuit R(λ)≡R:|0 [symbol] [symbol] |ψ(λ) [symbol] ≡|ψ [symbol] , that prepares an ansatz trial state, and a quantum circuit for implementing the projector Π:=I−2|0 [symbol] [symbol] 0|.

Three quantum registers are initialised to the states |+ [symbol] , |+ [symbol] , and |0 [symbol] respectively. To the third register, the preparation circuit is applied so that the registers are now |+ [symbol] , |+ [symbol] , |ψ [symbol] . The first register is the control register as used in the RFPE algorithm and the final two quantum registers are used in order to cast the expectation estimation subroutine as a RFPE problem. The quantum circuit S is defined as S:=S₀S₁ with S₀=(RΠR^{†}), S₁=(PRΠR^{†}P^{†}). Circuit S is depicted in FIG. 4. This circuit S is used in place of the U 310 in the RFPE algorithm so that after a rotation by an angle (Mθ) is applied to the first qubit, this then controls the operation of S on the second and third registers. Finally, the first register is measured in the Pauli-X basis.

Proposition 1

The operator S:=S₀S₁ with S₀=(RΠR^{†}), S₁=(PRΠR^{†}P^{†}) is a rotation by an angle ϕ=2 arccos(| [symbol] ψ|P|ψ [symbol] |) in the plane separated by |ψ [symbol] and |ψ′ [symbol] :=P|ψ [symbol] . Therefore, the state |ψ [symbol] is a superposition of eigenstates of S with eigenvalues e^{±iϕ} (i.e. eigenphases ±ϕ) and | [symbol] ψ|P|ψ [symbol] |=cos(±ϕ/2) can be estimated to a precision ∈ by running QPE on |ψ [symbol] to precision 2∈.

The operator S is physically implemented using a quantum circuit as depicted in FIG. 4. The quantum circuit of FIG. 4 comprises operators P, R, and H. The skilled person would be aware that the quantum gate H shown in FIG. 4 is a Hadamard gate which maps the basis state

$$\vert 0 〉 \to \frac{1}{\sqrt{( 2 )}} ( \vert 0 〉 + \vert 1 〉 ) and \vert 1 〉 \to \frac{1}{\sqrt{( 2 )}} ( \vert 0 〉 - \vert 1 〉 .$$

The quantum gate P of FIG. 4 represents a summand for which the expectation value is to be determined/estimated, for example corresponding to a tensor product of Pauli operators. The quantum gate R of FIG. 4 represents the arrangement of quantum circuits that are used to prepare the ansatz trial state. The dagger notation refers to a Hermitian conjugate so that P^{†} and R^{†} refer to the quantum gates corresponding to the Hermitian conjugate of P and R respectively. The quantum circuit S, that is constructed to operate on the ansatz trial state |ψ(λ) [symbol] , is therefore based on the arrangement of quantum gates that were used to prepare the ansatz trial state. The proposition shows that the quantum circuit, S, can be used to obtain useful information about the unknown quantity.

There are a range of arrangements of quantum gates that can be used to prepare the ansatz trial state |ψ [symbol] . For example, the Unitary Coupled Cluster ansatz is a powerful set of ansatz states that could be efficiently prepared in the circuit but for which there is no efficient classical method for calculating the desired expectation value.

In the α-QPE expectation estimation routine, the quantum circuit S is applied to the quantum circuit 300 of FIG. 3, replacing U in the known circuit depicted at 310. The α-QPE expectation estimation routine will be described later with reference to the flowchart of FIG. 9; step 908 of FIG. 9.

That S is a rotation may be seen by S₀=I−2|ψ [symbol] [symbol] [symbol] ψ| and S₁=I−2|ψ′ [symbol] [symbol] ψ′| and noting these are reflections across planes perpendicular to |ψ [symbol] and |ψ′ [symbol] respectively. The controlled S gate needed in phase estimation can be written as c−S=R(c−Π)R^{†}PR(c−Π)R⁵⁵⁴ P^{†} instead of adding controls on each unitary.

While [symbol] ψ|P|ψ [symbol] is guaranteed to be real, performing the algorithm as in Proposition 1 does not allow the sign to be discerned. This is fixed by instead estimating the amplitude

$$A \text{:} = \vert 〈 + ψ \vert c - P \vert + ψ ) \vert = \vert \frac{1 + 〈 ψ \vert P \vert ψ 〉}{2} \vert$$

where |+ [symbol] =½(|0 [symbol] +|1 [symbol] ) using the same method. Since −1≤ [symbol] ψ|P|ψ [symbol] ≤1, we can obtain it from A. FIG. 4 illustrates the circuit implementing c−S′ where S′ is the S necessary to compute A as per the proposition. Simply implementing α-QPE instead of QPE in the above casts expectation estimation into α-QPE as desired.

Since P is constructed from tensored Pauli matrices and Z=HXH and Y=iXHXH, C−P adds a cost of O(1) c−X≡c−NOT gates per Pauli gate, leading to O(n) c−NOT gates per P for an n-qubit Hamiltonian H with circuit depth O(n). Using a space overhead of O(n) and a binary tree, the c−P gate can be implemented with O(log(n)) circuit depth. The C H gate is an n-qubit controlled sign flip, an operator also used in Grover's algorithm, and is equivalent in cost (up to ˜2n single qubit gates with O(1) depth) to an n-bit Toffoli gate. While it is known that the circuit model implementation of the n-bit Toffoli gate requires at least 2n c-NOT gates, the best known implementation requires 32n−96 elementary gates. There is also a constant factor overhead for state preparation in the present approach: this means two R and two R†≡R−1 gates are needed.

Therefore, casting expectation estimation as α-QPE results in an overhead of O(n) single qubit gates and O(n) c NOT gates, with total circuit depth O(n), for each Pᵢ in the original VQE. The original implementation of expectation estimation in VQE requires O(n) single qubit gates and zero C−NOT gates with total circuit depth D=O(1). It is possible to tally an overhead on state preparation: two R and two R⁵⁵⁴ ≡R⁻¹ gates are needed. This overhead should be acceptable for example when R prepares the “device ansatz” which by definition means R is straightforward to implement accurately on a given device.

Conversely, implementing all circuits involving c−S′ᵢ, one for each Pᵢ sub-term in H, is more straightforward than faithfully implementing c−exp(−iHt) as typically required in QPE. Consider the typical case when H is the electronic Hamiltonian, written in second quantised form as:

$$H = {\sum }_{p q} {h}_{p q} {a}_{p}^{†} {a}_{q} + \frac{1}{2} {\sum }_{p q r s} {h}_{p q r s} {a}_{p}^{†} {a}_{q}^{†} {a}_{r} {a}_{s} ,$$

where the indices run over n introduced spin basis orbitals. With second order Trotter decomposition, implementing c−exp(−iHt) for fixed t requires, at first count, a circuit depth of O(n¹¹) as follows: O(n⁴) from the number of sub-terms in the second quantised form of H O(n) from the Jordan-Wigner transformation of these sub-terms necessary to preserve Fermionic commutation relations, and O(n⁶) from the Trotter decomposition. In recent years, rapid progress has been made in reducing the O(n¹¹) depth scaling, however the current best scaling with depth

$$\tilde{O} ( {n}^{\frac{8}{3}} )$$

using Taylor series based simulations is still worse than the best known depth for variational methods of O(n), which is asymptotically unaffected by the additive depth overhead of O(n) incurred.

It is important to note that circuit depth directly relates to coherence time which is a key quantum resource based on quantum superposition that is interchangeable with other quantum resources such as entanglement. Hence it is justified to base the comparison with QPE on circuit depth. In particular, even though O(n⁴) circuits need to be implemented involving c−S′ᵢ, one for each Pᵢ sub-term in H, the cost does not relate to increased quantum resources, but instead relates to increased repetitions using the same constant quantum resources. This strongly contrasts with the O(n⁴) additional circuit depth in implementing c−exp(−iHt) also coming from writing H as O(n⁴) sub-terms.

Tunable Bayesian QPE (α-QPE)—a Flowchart

A flowchart showing an implementation of α-QPE is given in FIG. 9. As will be appreciated, the method comprises an iterative method, routine and/or sub-routine. The method may be described as an algorithm for determining, or estimating, an expectation value of a summand. The summand is one of the summands which, when added together, provides a description of the energy level of interest of the physical system. The method shown in FIG. 9 is performed for each of the summands respectively. As discussed above, each summand comprises a different respective Pauli operator.

At step 900, the following parameters are inputted into the method: R, P, T and ∈. R is the preparation circuit R(λ):|0 [symbol] →|ψ(λ) [symbol] of the trial state |ψ(λ) [symbol] . P is the Pauli operator of the summand in question, i.e. Pᵢ≡P. T is the coherence time of the quantum computer and/or quantum processor 1102 being used to determine the expectation value of the summand. ∈ is the required error in the output as an estimate of ψ(λ)|P|ψ(λ). In other words, ∈ represents the required accuracy in the estimation of [symbol] ψ(λ)|P|ψ(λ) [symbol] .

In more detail, the preparation circuit R(λ):|0 [symbol] →|ψ(λ) [symbol] prepares the trial state |ψ(λ) [symbol] on the quantum computer and/or processor using an arrangement of quantum gates. A suitable arrangement of quantum gates is depicted in FIG. 4 and is described above.

At step 902, S is set to S(R,P). S is the circuit given in FIG. 4, without the control qubit on the top wire. α is set to α(T,∈), and N is set to N (T, ∈).

In more detail, an initial quantum circuit S is prepared based on the arrangement of quantum gates that were used in the preparation circuit R. The initial quantum circuit S is also prepared based on the Pauli operator P of the summand in question.

At step 902, the complexity of the initial quantum circuit to be used in the sub-routine 916 is set, based on the coherence time, T, and the required error, ∈. More specifically, the complexity of the quantum circuit is set by:

$$α = ( min ( \frac{log ( T )}{log ( \frac{1}{ϵ} )} , 1 )$$

The complexity of the quantum circuit refers to the number of applications, M, of the quantum circuit S on the trial state.

Also at step 902, the number of iterations to be performed, N, of the sub-routine 916 is set, based on the coherence time, T, and the required error, ∈. More specifically, if α=1, the number of iterations of the summand expectation value determination sub-routine that are to be performed is set to:

$$N = 4 log ( \frac{1}{ϵ} )$$

Otherwise, if α<1, the number of iterations of the sub-routine is set to:

$$N = 2 \frac{{( \frac{1}{ϵ T} )}^{2} - 1}{1 - log ( T ) \text{/} log ( \frac{1}{ϵ} )}$$

At step 904, certain parameters are initialised so as to provide initial values for the iterative sub-routine. The following parameters are initialised to the following values:

n=0;μ=0;σ=1

where μ is the algorithm's current estimate of ϕ. In other words, μ is the algorithm's current estimate of the phase ϕ of the trial state ω(λ). μ is iteratively updated as the algorithm progresses. σ is the algorithm's current estimate of the error in μ. n is a counter that increments after each iteration at step 914. In other words, n is a counter for the number of iterations of the sub-routine that have already been performed.

Blocks 906, 908, 910, 912 and 914 describe a summand expectation value determination sub-routine 916. The sub-routine 916 is performed N times in an iterative process, where N is set in step 902.

At step 906, the parameters M and θ are set by the following equations:

$$M = \frac{1}{{σ}^{α}} θ = μ - σ$$

where M determines the number of times the quantum circuit S is applied to the trial state |ω(λ) [symbol] . This is shown in FIG. 3, wherein U^{M} is replaced by S^{M} at 310 in FIG. 3, and so the quantum circuit S is applied to the trial state M times. In other words, M determines the complexity of the quantum circuit S that operates on the trial state |ψ(λ) [symbol] . In other words, M determines the coherence length requirement of the quantum circuit S, since S operates on the trial state M times. The quantum circuit S is depicted in FIG. 4 and the arrangement of this circuit is detailed above.

θ determines the rotation applied to the state |+ [symbol] on the top wire of the circuit in FIG. 3, at 302. |+ [symbol] represents the +1 eigenstate of the tensored Pauli X operator. More specifically, the circuit in FIG. 3 shows 302 which involves a rotation of Mθ on the top wire on the |+ [symbol] state.

Also at step 906, the algorithm generates a distribution [symbol] , wherein the distribution is based on μ and σ.

In more detail, the distribution [symbol] is a normal distribution, wherein the normal distribution is generated based on μ and σ. In yet more detail, the normal distribution [symbol] is generated at step 906. The mean of the distribution is determined by μ and the standard deviation of the distribution is determined by σ.

Within each iteration of the sub-routine 916, the values of μ and σ are updated at step 910. The distribution [symbol] generated at step 906 is generated at each new iteration of the sub-routine 916. In other words, a new distribution is generated at each iteration of the sub-routine 916. The distribution [symbol] generated at each new iteration is thus generated with respect to the updated values of μ and σ of the previous iteration.

At step 908, the quantum circuit 300 operates on the trial state |ψ(λ) [symbol] and the state |+ [symbol] , wherein U^{M} at 310 of FIG. 3 is replaced with S^{M}, wherein S is the quantum circuit shown in FIG. 4 (without the control qubit on the top wire). A measurement is made at 304 to produce a measurement value, E. In more detail, the measurement value E is made on the top wire of the quantum circuit shown in FIG. 3. The top wire involves a rotation by Mθ of the |+ [symbol] state on the top wire. The trial state applied to the bottom wire of the quantum circuit 300 involves M applications of the quantum circuit S shown in FIG. 4 (without the control qubit on the top wire). The measurement value E on the top wire of the quantum circuit 300 may be either a 0 or a 1.

At step 910, the values of μ and σ are updated based on the measurement value, E, obtained at step 908. In more detail, a new distribution [symbol] is generated based on the generation [symbol] generated in step 906, as well as the measurement value E obtained in step 908. In other words, the new distribution generated in step 910 [symbol] = [symbol] (E, [symbol] ).

In yet more detail, at step 910, the value of μ is updated by setting μ to be the mean μ′ of the new distribution [symbol] . The value σ is updated by setting σ to be the standard deviation σ′ of the new distribution [symbol] .

At step 912, the number of iterations n of the sub-routine 9191 that have already been performed is tested against the number of iterations N that need to be performed. If n>N, the algorithm proceeds to step 914. Otherwise, if n≥N, the algorithm proceeds to step 918.

In other words, if n>N, then the sub-routine has not been performed the required number of times, N, wherein N is set in step 902 and N is based on the coherence time, T, and the required error, ∈, as detailed above.

If n>N, the algorithm proceeds to step 914, wherein the counter for the number of iterations of the sub-routine that have already been performed is incremented by 1. The algorithm then proceeds to iterate the sub-routine 916 by returning to step 906. The parameters M and θ are updated at step 906 based on the updated values for μ and σ, determined at step 910 of the previous iteration. The distribution [symbol] is also updated at step 906 based on the updated values of μ and σ. The sub-routine proceeds 916 to repeat the steps 906, 908, 910, 912 as outlined above.

If n≥N, then the sub-routine 916 has been performed in an iterative manner at least the predetermined required number of times. The expectation value of the summand in question is determined or estimated at step 918 based on the mean μ of the distribution generated at step 910 of the previous or final iteration of the sub-routine. In more detail, the expectation value a or the estimate of the expectation value a of the summand in question is determined at step 918 using the equation

$$a = 〈 ψ \vert P \vert ψ 〉 = 2 cos ( \frac{μ}{2} ) - 1$$

In yet more detail, the error e in the estimation of the expectation value may be determined. The error e is set as the standard deviation σ of the of the distribution generated at step 910 of the previous or final iteration of the sub-routine.

At step 920, the algorithm outputs the expectation value or estimate of the expectation value a= [symbol] ψ|P|ψ [symbol] of the summand in question.

Generalised VQE—a Schematic Diagram

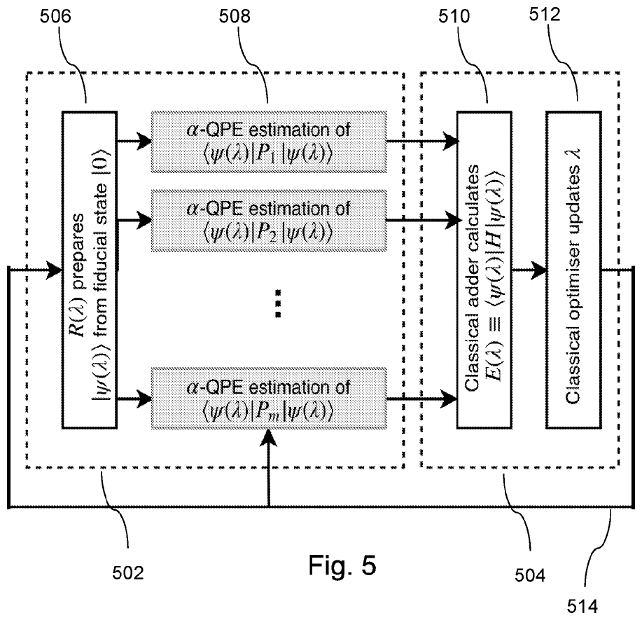

With reference to FIG. 5, a schematic of a new method of determining and/or estimating the energy level of a physical system, wherein the energy level may be described by the summation of a plurality of summands, is shown. The new method may be referred to as the generalised Variational Quantum Eigensolver (VQE) approach. Dashed box 502 depicts those parts of the method which are performed using a quantum computer, using quantum circuits. Dashed box 504 depicts those parts of the method which are performed using a classical computer, using classical circuits. Arrows between dashed boxes 502 and 504 depict the interface between the quantum and classical computers.

The Generalised Variational Quantum Eigensolver comprises an energy estimation routine that comprises steps 506, 508, 510 and 512 that are performed in an iterative process.

The preparation of the initial trial state is shown at box 506 of FIG. 5. At 506, a preparation circuit that uses an arrangement of quantum gates, R, comprised within the quantum computer is used to prepare an ansatz trial state |ψ(λ) [symbol] . This corresponds to at least one process of step 900 of the algorithm flowchart shown in FIG. 9, wherein the preparation circuit R(λ):|0 [symbol] →|ψ(λ) [symbol] prepares the trial state |ψ(λ) [symbol] on the quantum computer and/or processor using an arrangement of quantum gates.

At 508, the α-QPE algorithm of FIG. 9 is performed to determine or estimate the expectation value of each summand of the plurality of summands that describe the energy level of the physical system.

The α-QPE algorithm performed at step 508 is performed using a quantum computer. The quantum computer may determine parameters α and N at step 902 based on the coherence time, T, of the quantum computer and the required error, e. The quantum computer may generate the distribution [symbol] at step 906 based on the values μ and σ, for each iteration of the sub-routine 916. The quantum computer may determine the parameters M and θ at step 906 based on the values μ and σ, for each iteration of the sub-routine. The quantum computer may construct the quantum circuit 300 that operates on the ansatz trial state |ψ(λ) [symbol] at step 908 for each iteration of the sub-routine 916. The quantum computer may perform the measurement at step 908, (304 of the quantum circuit shown in FIG. 3) to obtain a measurement value [symbol] . The quantum computer may generate the new distribution V at step 910, based on the measurement value E obtained at step 908 and the distribution [symbol] generated at step 906, for each iteration of the sub-routine 916. The quantum computer may determine updated values for μ and σ based on the mean and standard deviation respectively of the new distribution [symbol] generated at step 910 for each iteration of the sub-routine 916.

The quantum computer may iterate the sub-routine 916 N times to determine or estimate the expectation value for one of the summands of the plurality of summands. In more detail, the quantum computer may determine or estimate the expectation value of each summand by determining the mean μ of the distribution [symbol] generated at step 910 of the final iteration of the sub-routine 916. In yet more detail, the quantum computer may determine or estimate the expectation value of each summand by determining the mean μ and applying this to the equation outlined above and at step 918 of FIG. 9. The quantum computer may then output the expectation value or estimate of the expectation value of the sub-routine at step 920.

The quantum computer may perform step 508 comprising the α-QPE estimation routine for the expectation value of each summand in parallel. In other words, the expectation value of one summand may be determined or estimated at step 508 at the same time as at least one of the other summands. The advantage here is to save time by determining or estimating the expectation value for as many summands as possible simultaneously.

The expectation value of each summand of the plurality of summands is communicated to a classical computer 504. The classical computer 504 sums the expectation values determined or estimated on the quantum computer at step 508 for each summand to determine an estimate for the trial state energy, E(λ).

In this embodiment, the expectation values are summed using a classical adder on a classical computer, however in another embodiment the summation of the expectation values may be performed on a quantum computer.

At step 512, an optimisation process is performed to update the trial state variable λ based on the energy estimate for the previous ansatz trial state. The updated trial state variable is communicated back to the quantum computer 502 such that the energy estimation routine is performed again, starting at step 506, wherein the quantum computer prepares the new ansatz trial state using a new arrangement of quantum gates, and wherein the new ansatz trial state is based on the updated trial state variable.

The Nelder-Mead (NM) method is an example of an algorithm that minimises a function by an iterative process. At each iteration, the function value is evaluated at the vertices of a simplex. The simplex is then evolved so that it iteratively shrinks to a single point—at which point the function takes its minimum. One key benefit of NM is that it does not require the function gradient at the simplex vertices which can be expensive for a quantum computer to provide. It is known that there are several alternative gradient-free algorithms (TOMLAB/GLCLUSTER, TOMLAB/LGO, and TOMLAB/MULTIMIN) that have been demonstrated to achieve the same accuracy with fewer function evaluations. Furthermore, it is expected that algorithms specifically designed to minimise a stochastic function (as appropriate in VQE) may reduce function evaluations further.

In this embodiment, the optimization process is performed using a classical computer, however in other embodiments the optimization process may be performed using a quantum computer.

Generally, the optimisation method/procedure can be thought of as acting to update the trial state variable so as to bring the trial state energy of the next ansatz trial state closer to the energy level of the physical system. As described above, on a first time the energy estimation routine is performed, the trial state is prepared using the Hamiltonian of the physical system and/or knowledge of the possible states which may be efficiently prepared using the quantum computer. As set out above, the optimisation procedure may comprise repeating the energy estimation routine a plurality of times in an iterative process to determine the energy level of the physical system. The optimisation procedure determines a new trial state variable to be used in the next iteration of the energy estimation routine. The optimisation procedure may be realized on a classical computer 1150, which then instructs a quantum computer 1110 to prepare the next state.

The energy estimation routine outlined above is performed a plurality of times in an iterative manner. During each iteration, the optimization process updates the trial state variable to be used to prepare the trial state for the next iteration. The energy estimation process is performed a plurality of times for a plurality of different trial states to determine a plurality of respective trial state energies.

In one embodiment, the energy level of the physical system may be determined by identifying the lowest trial state energy of the plurality of trial state energies.

VQE is generalised by replacing each expectation estimation routine for each summand in the standard VQE (shown in FIG. 2) with the α-QPE expectation estimation routine shown in FIG. 9. The casting ensures the ansatz trial state |ψ [symbol] is an eigenstate of the operator S which means that at each iteration of α-QPE |ψ [symbol] can be discarded, and a new state can be prepared and used. The discard-ability of |ψ [symbol] means the use of α-QPE, even when α=1, is different from the typical use of QPE when the input state is in superposition of eigenstates and cannot be discarded at each iteration. This point importantly justifies the formula for maximum depth D in (21) which is less than when not discarding |ψ [symbol] . A schematic of the generalised VQE is given in FIG. 5.

Generalised VQE still preserves the advantages of standard VQE distinct from decreased evolution time. For example, it is only necessary to estimate expectations of Pauli operators, which requires much less circuit depth to implement than exp(−iHt), as already discussed. Also, robustness via self correction is preserved because generalised VQE is still variational meaning it could still give accurate results without quantum error correction. Also, parameters to prepare the variational ansatz |ψ(λ) [symbol] at each optimisation iteration may be classically stored.

Additional Comments

The use of an iterative process within a summand expectation value determination sub-routine has never been considered within the framework of VQE, let alone implemented. The use of an iterative process in the manner described within the context of a quantum computer often increases the circuit depth requirements and hence requires a quantum computer having a longer coherence time. The prevailing thinking amongst researchers using VQE is that coherence time requirements should be reduced as far as possible so as to maximise the usefulness of VQE on today's quantum computers. Thus, a large number of identical, short circuits are used. In sharp contrast, the present method comprises performing a summand expectation value determination sub-routine a plurality of times in an iterative process. In some embodiments, each iteration of the summand expectation value determination sub-routine also comprises constructing a new quantum circuit based on the coherence time of the quantum computer and/or processor. With future quantum processors that will have longer coherence times, the energy levels of increasingly complex physical systems, for example larger molecules, can be probed. This type of iterative process within the sub-routine has never been considered before within the framework of VQE methods, and in fact goes against the current direction of VQE research.

Until the presently disclosed method, it was not known how to gain useful information from the increased coherence time in the context of the VQE algorithm. The current prevailing thought is that because the state |ψ(λ) [symbol] , is not an eigenvector of the measurement operator, Pᵢ, the only way to learn information is via statistical sampling. The present method shows that by modifying both the quantum state preparation and measurement operator together, an increase in the coherence time can result in significantly reduced runtime.

Another key algorithmic gain of the generalised VQE is the freedom to choose from a continuous range of regimes between statistical sampling and phase estimation.

Indeed neither edge regime is typically ideal: statistical sampling requires N=O(1/∈²) repetitions whereas phase estimation requires D=O(1/∈) coherence time. Each of these two regimes has been criticised by researchers using the other regime in exactly this way. The generalised VQE can directly answer such criticisms by optimally choosing α to trade off N and D, according to given costs on each. As is explained above, α is a factor determined based on the coherence time of the quantum computer and the required accuracy in the measurement.

The ability to discard and create new quantum circuits in the summand expectation value determination sub-routine, each newly created circuit having a complexity based on the available coherence time and the required accuracy in the estimate, means that full advantage is taken of available resources. This in turn reduces the time taken to determine the state energy. The ability to base the complexity of the quantum circuits comprised within each iteration of the summand determination sub-routine on the coherence time of the quantum computer is particularly important when one considers the speed with which the field of quantum computing is developing. It is envisaged that new quantum computers with longer coherence time will be produced as the field and corresponding technology develops. The present method will allow the speed and accuracy with which experimenters and scientists can probe the energy levels of a physical system to keep up with the pace of technological improvement, and in particular to allow researchers to make the most of available coherence times. On the other hand, the α-QPE is of independent theoretical interest in understanding the relationship between quantum (D) and classical (N) resources. Moreover α-QPE maps to a continuous transition between the standard quantum limit (α=0, ∈=0(1/\sqrt{N})) and Heisenberg limit (α=1, ∈=O(1/D)) in quantum metrology which further clarifies these two limits, in particular the confusion between N and D in them.

Similarly, making use of the same arrangement of quantum gates, R, in producing the new quantum circuits for each iteration of the summand expectation value determination sub-routine is beneficial because it provides greater efficiencies in the method, reducing the time required to construct and implement new quantum circuits and different arrangements of quantum gates, thus further reducing the time required to determine the state energy level of the physical system.

The design of the methods of the present disclosure are motivated by technical considerations of the internal functioning of a quantum computer. In particular, in view of the constraints of modern day quantum computers such as the maximum available coherence time, the present methods include constructing quantum circuits within the quantum computer with a complexity that depends on the coherence time of the computer, in order to maximally exploit the available coherence time to determine an energy level of a physical system.

Reference is made herein to an energy level of a physical system. The physical system could be any of an atom, a molecule, a collection of atoms, enzyme or part thereof, chemical, or material such as a potential superconductor. In each case, the energy level plays a central role in elucidating the properties of the chemical structures and reactions and as such has many applications in materials design, the design of new pharmaceuticals, or the design of novel catalysts.

In the search for new pharmaceuticals, the binding energy between the candidate drug and a target protein can be obtained from the methods of the present disclosure. This binding affinity is routinely used in the screening for candidate molecules as it is used to test if the molecule has the desired effect.

In the exploration of crystalline materials, the physical system corresponds to a bulk or surface of the material for example, composed of Lithium Ions (Li-Ions). The electric structure that can be derived by using the energy level of the system in order to design a material with specific properties. For example, the energy level is used to optimize the properties of the electroactive crystals in the design of better Li-ion batteries.

The high level of accuracy that results from the methods disclosed herein enables the calculation of the energetics of reaction intermediates and the kinetic barriers between molecules involved in a chemical reaction. This ability to predict and tune the reaction conditions enables the design of fast and energy efficient catalysts for applications such as the production of ammonia for use in fertiliser.

In addition, many other problems can be solved by mapping to a Hamiltonian and solving by finding an energy level such as the ground state. For example, optimisation problems as diverse as scheduling tasks or searching for faults in a circuit can be effectively solved by this method. As will be understood by the skilled person, an energy level of a physical system refers to the eigenvalues of the corresponding Hamiltonian.

To give an example of the many industrial applications of the present method, the search for a more efficient means of producing fertiliser is an example of a technological problem which could be aided by better understanding of reactant energy levels. The production of ammonia via the Haber-Bosch process is crucial for fertiliser production, but requires both high pressure and high temperatures and as a result is a very energy intensive process. Nitrogenase, in contrast, is an enzyme that achieves the same task at room temperature and at standard pressure, and there is therefore intense interest in understanding the nitrogenase enzyme. It is known that greater knowledge of the energy levels of the iron-molybdenum cofactor (FeMo-co) within the MoFe protein contained in the Nitrogenase enzyme would lead to significant advances in the discovery of a more efficient method for producing ammonia.

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

It will be understood that the above description of specific embodiments is by way of example only and is not intended to limit the scope of the present disclosure. Many modifications of the described embodiments are envisaged and intended to be within the scope of the present disclosure.

The above implementations have been described by way of example only, and the described implementations and arrangements are to be considered in all respects only as illustrative and not restrictive. It will be appreciated that variations of the described implementations and arrangements may be made without departing from the scope of the invention.

### Mathematical Appendices

Derivation of N and D for α-QPE

For a normal prior ϕ˜ [symbol] (μ,σ²), it is possible to calculate the expected posterior variance r² (i.e. the Bayes risk) by the formula

$${𝔼}_{E} [ 𝕍 [ φ \vert M , θ ; μ , σ ] ] ≡ {r}^{2} ( M , θ ; μ , σ ) ≡ {r}^{2} ( M , θ ) ≡ {r}^{2} = {σ}^{2} ( 1 - \frac{{M}^{2} {σ}^{2} {sin}^{2} ( M ( μ - θ ) )}{{e}^{{M}^{2} {σ}^{2}} - {cos}^{2} ( M ( μ - θ ) )} ) , \qquad ( 1 )$$

The variance, r², is bounded from below by an envelope σ²(1−M²σ²e^{M}^{²}^{σ}^{²}) which is minimised at:

$$M = \frac{1}{σ} , \qquad ( 2 )$$

However, this may be far away from the minimising M of r²(M,θ) due to oscillations of r², as a function of M, above this envelope. The rate of these oscillations is controlled by θ. It is understood that the optimal θ≈μ±σ “washes out” these oscillations, thereby aligning r² closer to its envelope. In the appendix below, the choice for optimal M and θ is justified to be of the form

$$M ∝ \frac{1}{σ}$$

and θ=μ±σ.

For θ=μ±σ, M=a/σ is used as a trial with a∈ [symbol] to give:

$$\begin{matrix}{r}^{2} ( \frac{a}{σ} , μ \pm σ ) = {σ}^{2} ( 1 - g ( a ) ) , where \text{:} & ( 3 ) \\ g ( a ) := \frac{{a}^{2} {sin}^{2} ( a )}{{e}^{{a}^{2}} - {cos}^{2} ( a )} . & ( 4 )\end{matrix}$$

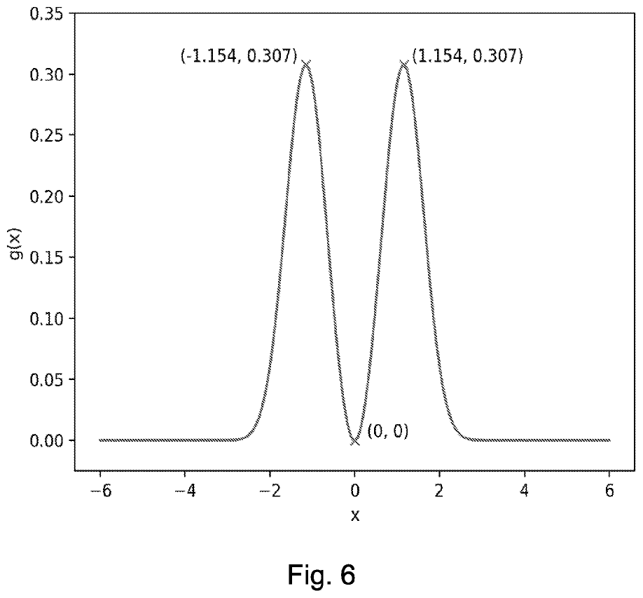

It is possible to show, that g is maximised at a=a₀≈±1.154 taking maximum value gₙₘₓ=0.307; a plot of g is given in FIG. 6. So r² is minimised at a=a₀ taking minimum value:

rₘᵢₙ²=k₀²σ², (5)

where k₀²≈0.693. This means that, after each iteration of RFPE, the variance is expected to (at least) decrease by a factor of k₀² when M and θ are chosen optimally, as detailed in the Appendix below.

Writing σₙ for the standard deviation at the n-th iteration, (5) is rewritten as [symbol] [σₙ²|σₙ₋₁²]=k₀²σₙ₋₁². Taking expectation over σₙ₋₁, the Law of Iterated Expectation gives:

[symbol]

[σₙ²]=k₀² [symbol] σₙ₋₁²]. (6)

Assuming that for n≥n₀ (some n₀ sufficiently large) [symbol] [σₙ]≡ [symbol] [(σₙ− [symbol] [σₙ])²]=0, and commuting squaring with expectation in (6) gives:

[symbol]

[σₙ]=k₀⁽ⁿ⁻ⁿ^{₀}⁾ [symbol] [σₙ_{₀}]. (7)

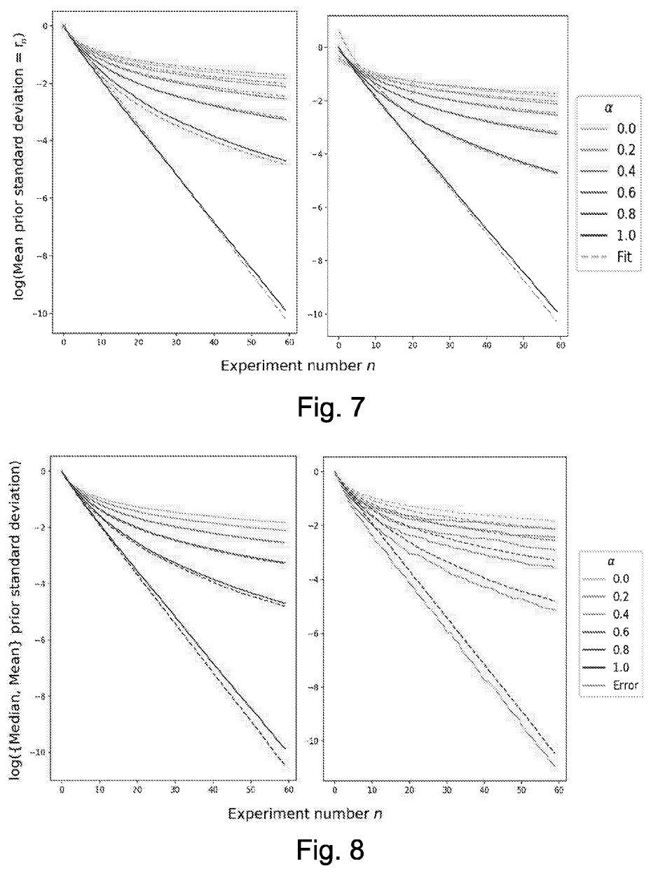

The small variance and subsequent assumptions/approximations are justified by good agreement of the final results with numerical simulations, shown in FIGS. 7 and 8. Note that the accuracy of later approximations based on Taylor series expansions can be assessed via the order of expansion.

Writing rₙ:= [symbol] [σₙ] for the expected standard deviation at the n-th iteration, (7) can be rewritten as:

rₙ=k₀⁽ⁿ⁻ⁿ^{₀}⁾rₙ_{₀}, (8)

so the standard deviation is expected to decrease exponentially with the number of iterations of RFPE.

Since rₙ of RFPE decreases exponentially with n, the use of M∝1/σₙ at the n-th iteration means M is expected to increase exponentially with n. This means that RFPE is indeed in the phase estimation regime which still has the same problem of requiring an exponentially long coherence time in the number of bits of precision required.

The present invention addresses the problem of long coherence time by considering N,D regimes that lay continuously between phase estimation and statistical sampling.

Observe that RFPE uses M=O(1/σ) and is in the phase estimation regime, but if M=O(1) at each iteration, the statistical sampling regime is recovered. Instead, considering M of the form:

$$M = {a ( \frac{1}{σ} )}^{α} , \qquad ( 9 )$$

with an introduced α∈[0,1] and some a=a(α)∈ [symbol] facilitate a transition between the two regimes.

Substituting the near optimal θ=μ±σ, but M as (9), into (1), giving expected posterior variance:

$${r}^{2} ( {a ( \frac{1}{σ} )}^{α} , μ \pm σ ) = {σ}^{2} ( 1 - g ( b ) ) , \qquad ( 10 )$$

where b:=aσ^{(1-α)} and g is as defined above. If b=a₀, this give a=a₀(1/σ)^{(1-α)}, but a is independent of σ. From the graph of g (shown in FIG. 6), we see there is no natural way to define an optimal a=a(α) except when α=1. It is possible take a=a₀ (which is independent of α) but instead set a=1 for notational convenience. It is necessary for Taylor approximations and divisions by (1−α) to also assume α≠1 unless stated otherwise.

For σ small, and so b small:

$$g ( b ) = \frac{{b}^{2}}{2} + O ( {b}^{4} ) , \qquad ( 11 )$$

which can be substituted into (10) to give the following upon taking expectations and using earlier assumption that [symbol] [σₙ]=0 for large n:

rₙ₊₁²=rₙ²(1−½(rₙ²)^{1-α}) (12)

Setting xₙ:=rₙ² in (12) gives:

xₙ₊₁=xₙ(1−½xₙ^{1-α}), (13)

which is similar to a logistic map. Taking log gives log(xₙ₊₁)=log(xₙ)−½xₙ^{1-α} to O(xₙ^{1-α})²), which gives, upon writing lₙ=log(xₙ):

lₙ₊₁=lₙ−½e^{(1-α)l}^{ₙ}, (14)

Assuming existence of a differentiable function l=l(t) with l(tₙ)=lₙ where tₙ:=nh, substitute l into (14) to obtain:

$$\frac{l ( {t}_{n} + h ) - l ( {t}_{n} )}{h} = \frac{- {e}^{( 1 - α ) l ( {t}_{n} )}}{2 h} . \qquad ( 16 )$$

Take h small and assume the LHS of (15) is well approximated by a derivative. Solving the resulting differential equation under initial condition at (n₀,rₙ_{₀}) gives:

$$log ( {r}_{n} ) = log ( {r}_{{n}_{0}} ) - \frac{1}{2 ( 1 - α )} log ( 1 + {r}_{{n}_{0}}^{2 ( 1 - α )} \frac{1 - α}{2} ( n - {n}_{0} ) ) . \qquad ( 16 )$$

Assess (16) with respect to the recurrence (14) it intended to solve by substituting it back, giving:

$$0 = {l}_{n + 1} - {l}_{n} + \frac{1}{2} {e}^{( 1 - α ) {l}_{n}} = O ( {( \frac{1}{{( n - {n}_{0} )}^{2} + \frac{2}{1 - α} {( 1 / {r}_{{n}_{0}}^{2} )}^{1 - α}} )}^{2} ) . \qquad ( 17 )$$

This means that for n≥n₀, (16) is expected to improve as a solution to (14) as n₀ increases (and so rₙ_{₀} decreases).

Equations (16) and (8) are plotted (the latter for completeness but with k₀² reset to k₀²=0.708 corresponding to a=1) against numerical simulations of RFPE between iterations 0 to 90 with two initial conditions (n₀, rₙ_{₀})=(0,r₀:=1) and (20,r₂₀). The numerical simulations are shown in FIGS. 7 and 8 and show good agreement with the analytical (16) and (8). Note that (16) neatly reduces to the form of (8) in the α=1 limit but not exactly because of the inaccuracy of approximation (11) when α=1.

Finally, rearranging (16) gives:

$$\begin{matrix}n = 2 {( \frac{1}{{r}_{{n}_{0}}^{2}} )}^{1 - α} \frac{1}{1 - α} ( {( \frac{{r}_{{n}_{0}}^{2}}{{r}_{n}^{2}} )}^{1 - α} - 1 ) + {n}_{0} \\ = O ( \frac{1}{1 - α} ( {( \frac{1}{{r}_{n}^{2}} )}^{1 - α} - 1 ) ) , \\ = O ( f ( {r}_{n} , α )\end{matrix} \\ wherein \\ f ( r , α ) = \{ \begin{matrix}\frac{2}{1 - α} ( \frac{1}{{r}^{2 ( 1 - α )}} - 1 ) 0 \leq α < 1 \\ 4 log ( \frac{1}{r} ) α = 1\end{matrix} \qquad ( 18 )$$

and (9) gives:

$${D}_{n} : = {max}_{\{ \leq n iterations \}} ( M ) = \frac{1}{{r}_{n}^{α}} , \qquad ( 19 )$$

By setting (n,Dₙ,rₙ)↔(N,D,∈) shows that the expected number of samples used in the α-QPE algorithm scales as

$$O ( \frac{1}{1 - α} ( {( \frac{1}{{ϵ}^{2}} )}^{1 - α} ) - 1 )$$

Optimal M,θ

The optimality (in RFPE) of both θ≈μ±σ and the form M∝1/σ is justified using the argument below. Recall that the probability of measuring E=0 in the RFPE circuit is:

$${P}_{0} = P ( 0 \vert φ ; M , θ ) = \frac{1 + cos ( M ( φ - θ ) )}{2} . \qquad ( 20 )$$

In order to gain maximal information about ϕ, the range of P₀ has to uniquely and maximally vary across the domain of uncertainty in ϕ. The Bayesian RFPE conveniently gives this domain [symbol] D=(μ−σ,μ+σ) of uncertainty at each iteration. A naive domain on which the range of COS uniquely and possibly maximally varies is [0,π]. So it is desirable to control (M,θ) such that M( [symbol] −θ) is equal to [0,π], i.e.

$$\{ \begin{matrix}M ( μ - σ - θ ) = 0 , \\ M ( μ + σ - θ ) = π\end{matrix} , \qquad ( 21 )$$

This has solution:

$$( M , θ ) = ( \frac{π / 2}{σ} , μ - σ ) ,$$

which is not far from the optimal choice found in the appendix above. Intuitively, the slight discrepancy could only be due to [0,π] not being the domain on which cosine uniquely and maximally varies.

FIG. 6 shows a plot of

$$g ( x ) = \frac{{x}^{2} {sin}^{2} ( x )}{{e}^{{x}^{2}} - {cos}^{2} ( x )} .$$

As can be appreciated, g has maxima at ≈(±a₀=±1.154,0.307) and minima at (0,0). Near x=0, g(x)=x²/2+0(x⁴).

Optimal α-QPE

In an experimental setting, N is the number of state preparations or the number of measurements; while D is proportional to the maximum coherence time. Attention is now turned to the optimal α that should be chosen given restrictions or costs on N and D. If zero cost is associated with N but some cost is associated with D then it is clear that the statistical sampling regime is best. Conversely, if some cost is associated with N but zero cost is associated with D then the phase estimation regime is best.

A study is presented wherein the particular constraint that (1≤)D≤D₀ for some constant D₀, i.e. D cost is zero until some threshold when it becomes infinite. This is experimentally realistic with D₀ equal to the transverse coherence time T₂ but where the standard e^{-t/T}^{₂} model for T₂ coherence is approximated by a step-function in t that jumps from full coherance to zero coherance at t=T₂. This step-function approximation is made in order to facilitate the following analytical analysis.

If precision 0<∈<1 is required and N is to be minimized and (16) is assumed to be true. Using N=ƒ(∈,α) as above, N is a decreasing function of α. There fore the minimal N is attained at the maximal

$$α = {α}_{max} = min \{ \frac{log ( {D}_{0} )}{log ( 1 / ϵ )} , 1 \} ,$$

giving:

$${N}_{min} = N ( {α}_{max} ) = \{ \begin{matrix}4 log ( \frac{1}{ϵ} ) & if {D}_{0} \geq \frac{1}{ϵ} \\ \frac{2}{1 - \frac{log ( {D}_{0} )}{log ( \frac{1}{ϵ} )}} ( {( \frac{1}{ϵ {D}_{0}} )}^{2} - 1 ) & if {D}_{0} < \frac{1}{ϵ}\end{matrix} . \qquad ( 22 )$$

The important point here is the inverse quadratic scaling with D₀ in the second case: through α it is possible to use all the coherence time available to the quantum computer to reduce the number of iterations. Without α, and if D₀<1/∈, then the quantum computer is resorted to statistical sampling with:

$$N = 2 ( \frac{1}{{ϵ}^{2}} - 1 ) , \qquad ( 23 )$$

which can mean significantly more iterations compared to (22. This study explicitly prescribes an optimal α given a realistic form of costs on N and D. A flowchart of optimal α-QPE is presented in FIG. 9.

### APPENDIX: RFPE-WITH-RESTARTS

Recall that, (16) is assumed as true, requiring precision within 0<∈<1, considering the particular constraint that (1≤)D≤D₀ for some constant D₀, i.e. D cost is zero until some threshold when it becomes infinite, and it is desired to minimise N, i.e. N costs N. Here the N required by RFPE-with-restarts is calculated, assuming decoherence is detected immediately at which point RFPE switches from phase estimation to statistical sampling.

Now, 1≤1/rₙ≤D₀ gives a maximum of N₀=4 log(D₀) iterations in this phase estimation regime. For n>N₀, RFPE-with-restarts switches to statistical sampling with M held constant at D₀. (18) then gives (under change of variable rₙ↔D₀rₙ throughout the derivation) the minimum number of total iterations of RFPE-with-restarts as:

$${N}_{min}^{′} = \{ \begin{matrix}4 log ( \frac{1}{ϵ} ) & if {D}_{0} \geq \frac{1}{ϵ} \\ 2 ( {( \frac{1}{ϵ {D}_{0}} )}^{2} - 1 ) + 4 log ( {D}_{0} ) & if {D}_{0} < \frac{1}{ϵ}\end{matrix} . \qquad ( 24 )$$

Again, an inverse quadratic scaling is seen with D₀ in the second case. In fact, this is always advantageous over the Nₘᵢₙ in (26) of optimal α-QPE, i.e. Nₘᵢₙ′≤Nₘᵢₙ, with equality if D₀∈{1}∪[1/∈,inf). One way of seeing this is by writing D₀=1/∈^{β} where β∈[0,1) when 1≤D₀<1/∈, giving:

$$\begin{matrix}\frac{{N}_{min}^{′}}{{N}_{min}} = 1 - β - β ( 1 - y ) \frac{log ( 1 - y )}{y} \\ = 1 - β + β ( 1 - y ) {\sum }_{k = 1}^{∞} \frac{1}{k} {y}^{k - 1} \\ \leq L ,\end{matrix} \qquad ( 25 )$$

where y=1−∈^{2 (1-β)}∈(0,1) with equality iff β=0 (i.e. D₀=1).

While Nₘᵢₙ′≤Nₘᵢₙ, it is less clear is if the experiment time (as opposed to number) of RFPE-with-restarts is also less than optimal α-QPE should experiment time been regarded as proportional to the sum of the M used at each iteration. In any case, should RFPE-with-restarts outperform optimal α-QPE in all relevant ways, it is possible use RFPE-with-restarts in the generalised VQE algorithm and the analysis of α-QPE serves to elucidate RFPE's performance in the statistical sampling α=0 regime. From this, it is possible to see that any QPE procedure may be substituted into the framework of generalised VQE.

It is presented that the two equations (24), (22) can be interpreted as two tradeoff relationships between classical resources (N) and maximal quantum resources (D)

α-QPE Analytical Versus Numerical Precision

As can be seen from FIG. 7, equation (16) agrees well with numerical simulations of RFPE for different values of α. Each simulation was performed with 200 randomised values of the true eigenphase ϕ (over which the mean is taken) and 900 samples from the posterior at each iteration obtained by rejection filtering. The plots on the left and right figures use initial conditions (n₀,rₙ_{₀})=(0,r₀:=1) and (20,r₂₀) respectively. The fit through (20,r₂₀) is more accurate for n≥n₀—this is expected since rₙ decreases as n increases, which improves all approximations based on rₙ small.

α-QPE Precision Versus Accuracy

As can be seen from the graphs of FIG. 8, good agreement is shown between the mean prior standard deviation and median prior standard deviation (left). The latter agrees qualitatively but not quantitatively with the median error (right, note that the pink lines going from above to below correspond to α increasing. That the median errors appear to tend toward zero would be a consequence of the asymptotic consistency of μₙ. This fact does not preclude the mean errors (not plotted) not tending towards zero and in fact they do not.

1. A method for determining an energy level of a physical system using a quantum computer controlled by a classical computer, the energy level of the physical system being described by the summation of a plurality of summands; the method comprising performing an energy estimation routine comprising:
preparing, by the quantum computer, an ansatz trial state using an arrangement of quantum gates, the ansatz trial state having a trial state energy dependent on a trial state variable,
estimating an expectation value of each summand respectively, the estimating comprising constructing, by the quantum computer based on the arrangement of quantum gates, an initial quantum circuit to operate on the ansatz trial state and performing, by the quantum computer, a summand expectation value determination sub-routine a plurality of times in an iterative process;
the energy estimation routine further comprising summing, by the quantum computer or the classical computer, the expectation value estimates of each summand to determine an estimate for the trial state energy;
the method further comprising determining the energy level of the physical system by applying an optimisation procedure to the energy estimation routine, the optimisation procedure comprising iteratively updating, by the classical computer or the quantum computer, the trial state variable and performing the energy estimation routine a plurality of times to determine a respective trial state energy for each of a plurality of different ansatz trial states.

2. The method of claim 1, wherein each iteration of the summand expectation value determination sub-routine comprises constructing a new quantum circuit.

3. The method of claim 2, further comprising operating, by the quantum computer, the newly constructed quantum circuit on the ansatz trial state to obtain a measurement value associated with an estimate of the summand expectation value.

4. The method of claim 3, wherein the new quantum circuit in each iteration of the summand expectation value determination sub-routine is constructed based on the obtained measurement value.

5. The method of claim 2, wherein the quantum computer has an associated coherence time, T, and the new quantum circuit in each iteration of the summand expectation value determination sub-routine is constructed based on the coherence time.

6. The method of claim 2, wherein each iteration of the summand expectation value estimation sub-routine further comprises generating, by the quantum computer, a distribution based on the measurement value, and the iterative process comprises updating, by the quantum computer, the distribution with each iteration based on the mean and standard deviation of the distribution of the previous iteration.

7. The method of claim 6, wherein estimating the expectation value of each summand comprises determining, by the quantum computer, the mean of the distribution produced during a final iteration of the summand expectation value determination sub-routine, the sub-routine being performed a predetermined number of times.

8. The method of claim 1, wherein the summand expectation value determination sub-routine comprises:
operating, by the quantum computer, the quantum circuit on the trial state to obtain a value, μ, associated with the estimate of the expectation value of the summand;
determining, by the quantum computer, an error, σ, associated with the value associated with the estimate of the expectation value; and
constructing, by the quantum computer, a new quantum circuit based on at least one of the determined error, σ, and the current value of μ.

9. The method of claim 2, wherein the energy level of a physical system is determined to a required accuracy ∈, and the new quantum circuit in each iteration of the summand expectation value subroutine is constructed based on the required accuracy, ∈.

10. The method of claim 9, wherein the new quantum circuit in each iteration of the summand expectation value sub-routine is constructed with a complexity dependent on T and ∈, T being the coherence time associated with the quantum computer, and the dependence of the complexity of the new quantum circuit on T and ∈ is given by α, wherein:
$$α = ( min ( \frac{log ( T )}{log ( \frac{1}{ϵ} )} ) , 1 ) .$$

11. The method of claim 1, wherein the energy level is determined to a required accuracy, ∈, and the summand expectation value determination sub-routine is repeated N times, wherein N is dependent on ∈.

12. The method of claim 1, wherein the summand expectation value determination sub-routine is repeated N times, wherein N is based on a coherence time, T, associated with the quantum computer.

13. The method of claim 1, wherein determining the energy level of the physical system comprises identifying the lowest determined trial state energy.

14. The method of claim 1, wherein the trial state variable is updated so as to bring the trial state energy of the next ansatz trial state closer to the energy level of the physical system.

15. The method of claim 1, wherein, on a first time the energy estimation routine is performed, the trial state is prepared using the Hamiltonian of the physical system and/or knowledge of the possible states which may be efficiently prepared using the quantum computer.

16. The method of claim 1, wherein the optimisation procedure comprises repeating the energy estimation routine a plurality of times in an iterative process to determine the energy level of the physical system.

17. The method of claim 16, wherein the optimisation procedure determines a new trial state variable to be used in the next iteration of the energy estimation routine.

18. The method of claim 1, wherein each summand comprises an operator, optionally wherein the operator is a tensored Pauli matrix.

19. A computer readable medium comprising computer-executable instructions which, when executed by a quantum processor, cause the processor to:
prepare an ansatz trial state using an arrangement of quantum gates, the ansatz trial state having a trial state energy dependent on a trial state variable,
estimate an expectation value of each summand respectively, the estimating comprising constructing, based on the arrangement of quantum gates, an initial quantum circuit to operate on the ansatz trial state and performing a summand expectation value determination sub-routine a plurality of times in an iterative process.
